(19) United States
(12) Patent Application Publication
(10) Pub. No.: US 2026/0198852 A1
(43) Pub. Date: Jul. 16, 2026

(54) METHOD AND APPARATUS FOR CAPTURING FLIGHT TRAJECTORY INDUCED ZERO GRAVITY CONDITIONS

(71) Applicant: Olivas & Associates, Inc., Manhattan Beach, CA (US)

(72) Inventors: John D. Olivas, Manhattan Beach, CA (US); Chloe Hank, Houston, TX (US)

(73) Assignee: Olivas & Associates, Inc., Manhattan Beach, CA (US)

(21) Appl. No.: 19/026,312

(22) Filed: Jan. 16, 2025

(51) Int. Cl. A61B 5/00; A61B 5/0205; B64D 45/00; B64G 7/00

(52) U.S. Cl. CPC A61B 5/4884; A61B 5/02055; B64D 45/00; B64G 7/00; A61B 2560/0242

(57) ABSTRACT

A method and system of instruments are disclosed for characterizing conditions within an aircraft during a flight designed to minimize gravitational effects. Scientific research can require experimentation under very low G or zero G conditions, and such conditions can be achieved in an aircraft using a specially designed trajectory to mimic freefall. Certain data can be collected during the critical portion of the trajectory to assess atmospheric, biometric, and localized information to better assess the results of the experiments.

### BACKGROUND

The present invention relates to reduced gravity test environments, and more particularly to a method and collection of sensors to characterize the test environment for micro gravity research. The invention is particularly useful in connection with aircraft based test beds that achieve the desired environment through parabolic flight.

Since the inception of America’s human space flight program in the 1960’s, engineers and scientists have relied, in part, on the use of military and civilian aircraft to conduct experiments with a near weightless environment. This environment is also known as reduced g (gravity) or micro g. Through the use of a parabolic flight path, an aircraft can reach a sufficiently high altitude and then gradually pitch the nose down to obtain a rate of descent to simulate the desired reduced gravity envrionment. This rate, depending on the angle and speed of the aircraft, results in a condition within where the cargo and occupants experience a local acceleration that is akin to that of gravity environment less than 1, as typically experienced on earth while standing still. In an ideal flight trajectory, the aircraft exactly matches the acceleration of gravity (freefall), whereby the gravitational forces dissipate to replicate those conditions experienced in the Low Earth Orbit of space. Similarly, matching the gravitational acceleration of approximately 1/6^{th} and 1/3^{rd} g of the Moon and Mars Lunar and Martian gravities can also be simulated. For the purposes of research, the conditions within the aircraft may experience the desired gravitational environment during the descent for durations between a few to over 30 seconds, before the parabolic flight path ends and the trajectory levels off, pitches the aircraft nose up, then the parabolic flight path is repeated.

Because the aircraft is flown manually, it is the responsibility of the pilots to use the aerodynamic surfaces and engine thrust, to control the desired environment. However, external conditions, such as pressure density, winds aloft, temperature, aircraft performance, operator experience, and many other factors affect the ability of the cargo and occupants to experience exact gravitational environments. Furthermore, variation in hardware design, center of gravity, location within the aircraft, sensitivity, and many additional factors can impact the performance on any particular research objective. In the past, the quality of the test bed environment was largely a function of pilot experience and weather of the day. While gross measurements were provided to researchers through digital displays or flight crew call outs, researchers have been reduced to accepting whatever quality that can be obtained from any given parabolic flight, with no ability to determine if the experiment results were a function of the weather, pilot experience, experiment design, or an actual result or observation of the experiment. Since many research experiments require very exacting measurements and the conditions within the less than the approximate thirty seconds are critical, more is needed to advance the performance of this technique.

With the advent of compact, portable, and electrically isolated sensor technology, it is possible to create a sensor package that can both record and store data from a package or “suite” specifically designed for parabolic flight micro gravity test beds. Additionally, more cost effective solutions allow for a more robust and expanded data set to more fully analyze and characterize the entire flight profile, including the external and internal environment, aircraft, and test apparats.

Because acceleration is the key driving parameter for all, accelerations in the X, Y and Z directions should be captured. Additionally, attitude in pitch, yaw, and roll are of interest. An optimal sampling rate allows for sufficient granularity to allow researchers to make post flight performance analysis. This is accomplished by a companion sensor suite, or subset thereof, located on strategic locations of the experiment fixture or support structure. Post flight, comparisons can quickly be made through algorithms comparing the data captured by both suites of sensors to aid in determining what adjustments are needed. This allows the researchers to extract critical data on performance and quality of the micro gravity environment, performance of the aircraft and the experiment within the aircraft, and ultimately the resulting behavior of the objective of the experiment.

Moreover, other parameters, such as internal relative humidity and temperature can and do change throughout the parabolic flight sortie. Sorties in which data is collected can range from less than 10 per flight, to greater than 50. Peak altitudes can range from mid to high 30,000feet AGL followed by dives to altitudes in the mid to low 20,000 feet AGL while the external temperature variation can exceed a 60° F differential. These external conditions affect the internal temperature and pressure of the fuselage in which the research is conducted. As such, these parameters will be of importance to some researchers.

In some aircraft, the beginning of the micro g parabola occurs within the stratosphere while the recovery at the end of the dive occurs within the troposphere. This consequential dip in and out of the thicker part of the atmosphere results in fluctuating exposures in cosmic radiation. By including an active radiation detector in the sensor suite, the micro-Sieverts per hour can be measured and evaluated against the research objective performance.

While some experiments and tests conducted it in a micro g environment are not intended to interact with humans, many tested in this type of environment are destined to be an integral part of the human space flight. This includes tools, experiments, spacesuits, exercise equipment, hygiene equipment, personally worn devices and the like. Because no two individuals are identical, biometric sensors can be worn by participants to also understand the effects of the micro g environment on a variety of human physiological responses. This includes, but are not limited to, heart rate, respiration rate, perspiration, blood pressure, pulse oxygenation, stress level, and other factors. By including biometric data that corresponds to the data collected on parabolic quality, the effects of the environment can also be extended to a human subject as the vessel for the test or research objective, which may be a human response or the intimate response between the human and the equipment beign evaluated.

Finally, the fidelity of the sensor suite would benefit from incorporating available flight data collected and downloaded by the avionics system of the aircraft. This information may include altitude, air speed, gravity meter, heading, angle of attack, etc. This more integrated approach would provide critical pilot feedback to improve aircraft handling by focusing on flight based on test environment rather than solely on aircraft response.

### SUMMARY OF THE INVENTION

The present invention is a method and sensor package having sensors, including but not limited to:

a three-axis accelerometer capable of detecting gravitational fields from-3 to 3 g;

an inertial measurement sensor;

an in-cabin temperature detecting thermocouple;

a pressure sensor;

a cabin atmosphere trace gas sensor;

a relative humidity sensor;

a radiation sensor and

an ultrasonic vibration sensor.

The sensors are powered via a self-contained power supply, preferably a rechargeable battery. Data may be collected via Bluetooth, wifi, or locally stored for download, as well as displayed on a connected screen or directly on the sensor package. Signals collected by the powered sensors are passed through a signal conditioner and into a digital acquisition system. Depending on available power and microprocessor speed, processing of the signals can be performed locally within the sensor suite or raw data can be downloaded for detailed meshing and incorporation once the sortie is complete. This later method would allow for fusing of aircraft and biometric data if the sensor suite remains independent. Additionally, sensor suites incorporated directly into research payloads can be fused with onboard processors to allow for in situ flight analysis of experiment.

These and other features of the invention will best be understood with reference to the accompanying drawings and the detailed description of the invention set forth below.

### BRIEF DESCRIPTION OF THE DRAWINGS

FIG. 1 is a schematic diagram of the data processing path of the present invention;

FIG. 3 is a schematic diagram of the aircraft data unit;

FIG. 4 is a schematic diagram of the microgravity data suite;

FIG. 5 is a schematic of the science payload sensors; and

FIG. 6 is a schematic diagram of the biometric data unit.

### DETAILED DESCRIPTION OF THE PREFERRED EMBODIMENTS

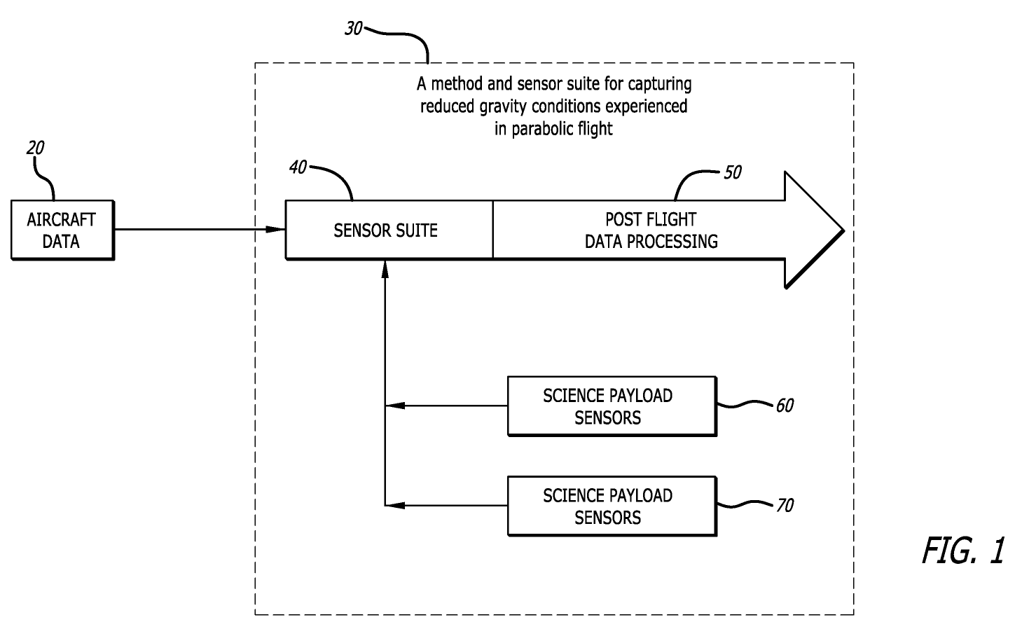

FIG. 1 shows the path for data to be processed by the present invention. When the aircraft is in flight, sensors read and develop signals corresponding to the aircraft flight data from flight data unit 20. This data is sent to and collected by the sensor suite 40 within the structure 30, which then forwards the processed (or raw) data to the post flight data processing unit 50. The sensor suite 40 also receives data directly from payload sensors 60 and biometric sensors 70, described in more detail below.

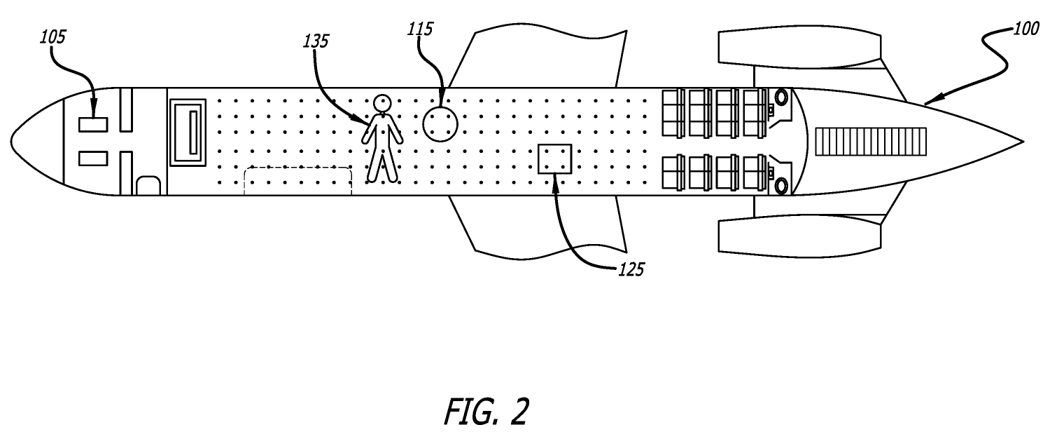

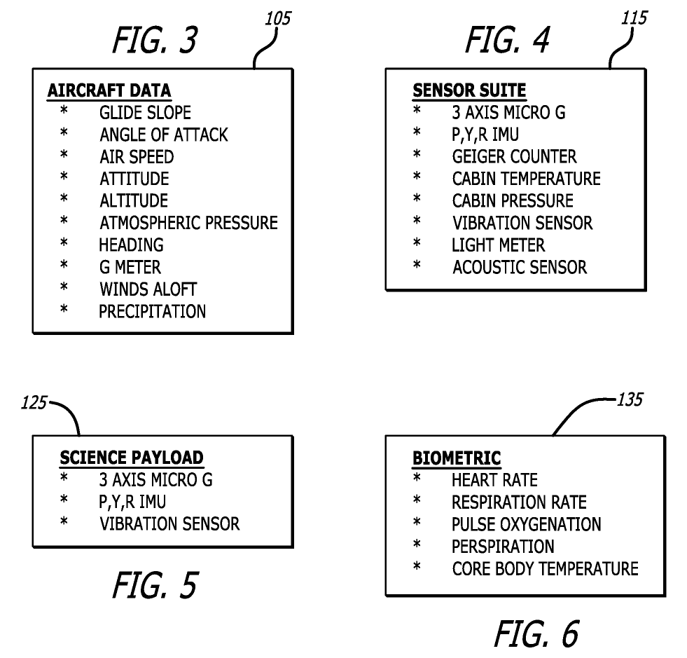

FIG. 2 is a layout view of an aircraft 100 incorporating the present invention. There are four data acquisition centers on the aircraft corresponding to the sensor packages of the present invention. Unit 105 represents the aircraft data unit and includes sensors that collect information on the flight conditions, including guide slope, angle of attack, air speed, altitude, attitude, atmospheric pressure, heading, gravitational meter, wind speed, and precipitation (see FIG. 3). Unit 115 represents the reduced gravity sensor suite, which includes sensors measuring 3 axis microgravity, radiation (Geiger counter), cabin pressure, cabin temperature, cabin trace gas, accelerometers for yaw, roll, and pitch, vibration in the payload bay, an acoustic sensor, and a light meter (See FIG. 4). Unit 125 corresponds to the science payload recordings, which include sensors for microgravity along all three axes, vibration, and yaw, roll, and pitch accelerometers (FIG. 5). Finally, unit 135 represents the biometric data unit that includes information relating to heart rate, respiration rate, pulse oxygenation, perspiration, and core body temperature (FIG. 6). Each of these units cooperate to give a complete picture for the aircraft’s interior conditions during a microgravity flight.

FIG. 3 is a diagram representing the systems flight data unit 105 typically located in the aircraft’s cockpit. The flight data unit 105 includes sensors for glide slope, angle of attack, air speed, attitude, altitude, atmospheric pressure, heading, gravity, wind speed and direction, and precipitation. This is a non-exclusive set of sensors that could be supplemented depending on the experiments to be performed and the specific conditions sought to be evaluated.

FIG. 4 corresponds to the microgravity sensor suite 115 that pertains particularly to conditions within a microgravity environment, including 3 axes microaccelerometers, pitch, yaw, and roll sensors, a Geiger counter, cabin pressure sensor, cabin temperature sensor, vibration sensor, light meter, and acoustic sensor.

FIG. 5 represents the science payload sensors unit scientific 125 and includes three axes micro g sensors, pitch, roll, and yaw sensors, and desired sensor subset, such as vibration sensors, temperature sensors, etc.

FIG. 6 represents the biometric data unit 135 and includes such sensors as a heart rate monitor, respiration rate sensor, pulse oxygenation sensor, perspiration monitor, core body temperature sensor, motion sensor, and other desired biologically induced measurement parameters.

Then the data from the foregoing data acquisition units are combined and processed, the conditions within the aircraft payload bay can be analyzed during a microgravity flight and multiple flights can be compared with the various conditions accounted for. This allows for better experimentation and analysis, and improves on any system heretofore conceived for microgravity flight.

While the foregoing describes the inventors’ preferred embodiments, it is understood that the invention is not limited to any specific embodiment or depiction in the figures. A person of ordinary skill in the art will readily recognize and appreciate various substitutions and modifications to the foregoing descriptions, and the invention should be considered to include all such modifications. Accordingly, the scope of the invention is properly determined by the wording of the appended claims using their ordinary meanings, consistent with but not limited to the preceding descriptions and depictions.

We claim:

1. A method for capturing a condition within an aircraft during a microgravity flight, comprising:
establishing an aircraft data collection unit configured for determining air speed, altitude, atmospheric pressure, heading, gravity, and acceleration;
establishing in a different location of the aircraft a microgravity suite of sensors, including accelerometers along three orthogonal axes, pitch, roll, and yaw sensors, a local pressure sensor, a local temperature sensor, a local radiation sensor, a vibration sensor, and an acoustic sensor;
establishing a biometric unit configured for determining heart rate, respiration rate, pulse oxygenation, and core body temperature of a user;
flying the aircraft on a parabolic flight to establish a microgravity condition;
collecting data from the aircraft data collection unit, the microgravity suite, and the biometric unit;
combining the data in the aircraft data collection unit with the microgravity suite to determine a condition within the aircraft during a microgravity flight; and
using the condition within the aircraft during the microgravity flight to assess a human using biometric data collected by the biometric unit.

2. A system for capturing a condition within a payload bay of an aircraft during a microgravity flight, comprising:
a first data source comprising an aircraft data collection unit configured for determining air speed, altitude, atmospheric pressure, heading, gravity, and acceleration;
a second data source comprising a data suite disposed in the aircraft payload bay, comprising accelerometers along three orthogonal axes, pitch, roll, and yaw sensors, a local pressure sensor, a local temperature sensor, a local radiation sensor, a vibration sensor, and an acoustic sensor;
a third data source comprising a biometric unit configured for determining heart rate, respiration rate, pulse oxygenation, and core body temperature of a user; and
a processor coupled to the aircraft data collection unit, the data suite, and the biometric unit, for onboard processing of data received from said first, second, and third data sources.

3. The system for capturing a condition of claim 2, wherein the data suite includes a light meter.

4. The system for capturing a condition of claim 2, wherein the aircraft data collection unit further comprises a precipitation sensor.

5. The system for capturing a condition of claim 2, wherein the biometric unit is located in the payload bay.

6. The system for capturing a condition of claim 2, wherein the data suite is located in the payload bay.

7. The system for capturing a condition of claim 2, wherein the aircraft data collection unit is located in a cockpit of the aircraft and is connected to the processor, said processor located in the payload bay.
